(19) United States
(12) Patent Application Publication
(10) Pub. No.: US 2026/0229354 A1
(43) Pub. Date: Aug. 6, 2026

(54) METHOD, PROGRAM, AND DEVICE FOR PROGNOSIS OF HEART FAILURE

(71) Applicant: MEDICAL AL CO., LTD., Seoul (KR)

(72) Inventors: Joonmyoung KWON, Seoul (KR); Hakseung LEE, Seoul (KR); Gain HAN, Seoul (KR)

(21) Appl. No.: 19/149,537

(22) PCT Filed: Mar. 13, 2024
(86) PCT No.: PCT/KR2024/095487

§ 371 Date: Jul. 19, 2025

(30) Foreign Application Priority Data
Mar. 13, 2023 (KR) 10-2023-0032857
Mar. 11, 2024 (KR) 10-2024-0033718

(51) Int. Cl. G16H 50/20; A61B 5/346; G06N 3/0464; G16H 50/30

(52) U.S. Cl. CPC G16H 50/20; A61B 5/346; G06N 3/0464; G16H 50/30

(57) ABSTRACT

According to one embodiment of the present disclosure, there is disclosed a method, program, and device for predicting the prognosis of heart failure that are performed by a computing device. The method may include: obtaining electrocardiogram data of a heart failure patient; and outputting a variable for the prediction of the prognosis of the patient based on the obtained electrocardiogram data by using a pre-trained deep learning model.

### TECHNICAL FIELD

The content of the present disclosure relates to artificial intelligence technology in the medical field, and more particularly, to a method for predicting the prognosis of a heart failure patient based on an electrocardiogram.

### BACKGROUND ART

Heart failure with reduced ejection fraction (HFrEF) poses a significant clinical challenge when its significant impact on global prevalence, morbidity, and mortality is taken into consideration. As the population ages, it is expected that the burden of HETEF increases and, thus, efficient prognostic tools to provide guidance on patient management and therapeutic decision-making are required. Despite significant progress in recent years, various studies have shown that the five-year survival rate for patients with HFrEF ranges from 53% to 67% and still remains far from optimal. Therefore, accurate risk stratification and prognostic prediction are of utmost importance to identify high-risk patients and tailor treatment strategies accordingly.

Although there have been studies that rely on conventional statistical approaches or existing predictive models, these methods have not been widely used due to their inherent limitations. Furthermore, although existing risk scores for heart failure have historical significance, they do not accurately reflect modern clinical practice because treatment protocols have evolved significantly since their initial development. The external validation of classical risk scores is performed using patient data that are up to 10 years old, so that there are raised questions about whether these results can be reliably extrapolated to modern patient populations. Furthermore, the application of these models is often hindered by the complex nature of the variables involved. The prognosis of patients with heart failure is influenced by a variety of factors, ranging from comorbidities at admission to variability in standard treatment and post-discharge treatment. Although many risk scores take into consideration factors such as admission laboratory tests and comorbidities, they may fall short of capturing all the variables that influence patient prognosis.

### DISCLOSURE

### Technical Problem

An object of the present invention is to provide a method of predicting the prognosis of heart failure based on electrocardiogram data by using artificial intelligence.

However, the objects to be achieved in the present disclosure are not limited to the object mentioned above, and other objects not mentioned may be clearly understood based on the following description.

### Technical Solution

According to one embodiment of the present disclosure for achieving the above-described object, there is disclosed a method of predicting the prognosis of heart failure that is performed by a computing device. The method may include: obtaining electrocardiogram data of a heart failure patient; and outputting a variable the prediction of the prognosis of the patient based on the obtained electrocardiogram data by using a pre-trained deep learning model.

Alternatively, the variable for prediction of prognosis may be the mortality of the patient within n years from the time when the electrocardiogram data was measured.

Alternatively, the deep learning model may include a combination of a stem block, residual blocks, and a fully connected network.

Alternatively, the deep learning model may be trained such that a negative predictive value satisfies a cutoff value determined using Youden's J statistic.

Alternatively, the deep learning model may receive clinical data of the patient together with the electrocardiogram data, and may output a variable for prediction of prognosis of the patient.

Alternatively, the clinical data may be a predictor selected through Cox regression analysis in a process of training the deep learning model. Furthermore, the clinical data may include at least one of pieces of information about age, sex, body mass index, blood pressure, heart rate, ejection fraction, whether chronic disease has been diagnosed, and whether optimal treatment has been performed.

Alternatively, the deep learning model may perform prediction while providing weight to first waveform features of at least one of V1 and V3 leads in the obtained electrocardiogram data.

Alternatively, the first waveform features may be ST segments.

According to one embodiment of the present disclosure for achieving the above-described object, there is disclosed a computer program stored in a computer-readable storage medium. The computer program causes operations for predicting the prognosis of heart failure to be performed when executed by at least one processor. In this case, the operations may include operations of: obtaining electrocardiogram data of a heart failure patient; and outputting a variable for the prediction of the prognosis of the patient based on the obtained electrocardiogram data by using a pre-trained deep learning model.

According to one embodiment of the present disclosure for achieving the above-described object, there is disclosed a computing device for predicting the prognosis of heart failure. The computing device may include: a processor including at least one core; memory including program codes executable by the processor; and a network unit configured to obtain electrocardiogram data of a heart failure patient. In this case, the processor may output a variable for the prediction of the prognosis of the patient based on the obtained electrocardiogram data by using a pre-trained deep learning model.

### Advantageous Effects

According to the method of the present disclosure, a variable for the prediction of prognosis, such as the mortality of a heart failure patient within a specific period, may be accurately estimated based on electrocardiogram data by using an artificial intelligence model, and high-risk patients may be effectively identified. Accordingly, the artificial intelligence model of the present disclosure may be effectively utilized as a decision making support tool for clinicians in heart failure management.

In addition, unlike conventional approaches, the artificial intelligence model of the present disclosure may be updated and improved over time, allowing it to be adjusted to treatment guidelines, clinical practices, and patient populations. This adaptability allows the artificial intelligence model of the present disclosure to maintain accuracy over time.

### DESCRIPTION OF DRAWINGS

FIG. 1 is a block diagram of a computing device according to one embodiment of the present disclosure;

FIG. 2 is a block diagram showing the analysis process of a deep learning model according to one embodiment of the present disclosure; and

FIG. 3 is a flowchart showing a method of predicting the prognosis of heart failure according to one embodiment of the present disclosure.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Furthermore, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “obtaining” used herein can be understood to mean not only receiving data over a wired/wireless communication network connecting with an external device or a system, but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Furthermore, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

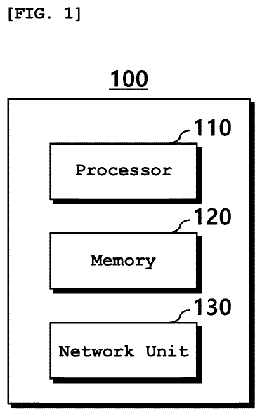

FIG. 1 is a block diagram of a computing device according to one embodiment of the present disclosure.

A computing device 100 according to one embodiment of the present disclosure may be a hardware device or a part of a hardware device that performs the comprehensive processing and computation of data, or may be a software-based computing environment connected over a communication network. For example, the computing device 100 may be a server that is a main agent for performing an intensive data processing function and sharing resources, or may be a client that shares resources through interaction with server. Alternatively, the computing device 100 may be a cloud system in which multiple servers and clients comprehensively process data while interacting with each other. Since the above description is only one example related to the type of computing device 100, the type of computing device 100 may be configured in various manners within a range understandable to those skilled in the art based on the content of the present disclosure.

Referring to FIG. 1, the computing device 100 according to one embodiment of the present disclosure may include a processor 110, memory 120, and a network unit 130. However, FIG. 1 is only an example, and the computing device 100 may further include other components for implementing a computing environment. Furthermore, only some of the disclosed components may be included in the computing device 100.

The processor 110 according to one embodiment of the present disclosure may be understood as a constituent unit including hardware and/or software for performing computing operations. For example, the processor 110 may read a computer program and perform data processing for machine learning. The processor 110 may process operation processes such as the processing of input data for machine learning, the extraction of features for machine learning, and the computation of errors based on backpropagation. The processor 110 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 110 described above are only examples, the type of processor 110 may be configured in various manners within a range understandable to those skilled in the art based on the content of the present disclosure.

The processor 110 may train a deep learning model to output a variable for the prediction of the prognosis of a patient whose electrocardiogram data was measured based on the electrocardiogram data. For example, the processor 110 may input heart failure data of a heart failure patient to the deep learning model. When the deep learning model analyzes the electrocardiogram data and predicts the possibility that the patient will die within a specific period, the processor 110 may calculate an error by cross-comparing a prediction result with death certification data related to the patient. In this case, the specific period is a period set by a user, and may be n years (n is a natural number) from the time when the electrocardiogram data was obtained from the patient. The processor 110 may update neural network parameters constituting the deep learning model based on the calculated error. The processor 110 may train the deep learning model by repeatedly performing this computational process so that the error is minimized. In addition to the supervised learning method described in the above-described example, the present disclosure may apply various learning methods, such as unsupervised learning and semi-supervised learning, depending on the structure or type of the deep learning model.

The processor 110 may estimate a variable for the prediction of the prognosis of a patient based on electrocardiogram data of the patient by using a pre-trained deep learning model. For example, when electrocardiogram data of a heart failure patient is measured, the processor 110 may predict the probability that the patient will die within a specific period from the time when the electrocardiogram data was measured based on the electrocardiogram data. In this case, the specific period may be a period determined by a user during the process of training the deep learning model. When the prediction of the deep learning model is completed, the processor 110 may generate a user interface for visualizing prediction results obtained through the deep learning model. The processor 110 may improve the accuracy of risk stratification through prognosis prediction using such a deep learning model, and may thus provide new insight into a variable for prognosis prediction. Furthermore, through such prognosis prediction using a deep learning model, the processor 110 may provide a medical environment that can provide customized treatment to patients in a timely manner, improve treatment results, and increase the trust of patients and medical staff in the treatment pathway, and may bring about innovation in the management of diseases such as heart failure.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize and manage the data required for the processor 110 to perform operations, combinations of data, and the program codes executable by the processor 110. For example, the memory 120 may store the medical data received via the network unit 130 to be described later. The memory 120 may store the program codes that operate a machine learning model to receive medical data as input and perform learning, the program codes that operate a machine learning model to receive medical data as input and perform inference according to the purpose of use of the computing device 100, and the processed data that is generated as the program codes are executed.

The network unit 130 according to one embodiment of the present disclosure may be understood as a constituent unit that transmits and receives data via any type of known wired/wireless communication system. For example, the network unit 130 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 130 may be applied in various manners other than the above-described examples.

The network unit 130 may receive data, required for the processor 110 to perform operations, through wired/wireless communication with any system, any client, or the like. Furthermore, the network unit 130 may transmit data, generated through the operations of the processor 110, through wired/wireless communication with any system, any client, or the like. For example, the network unit 130 may receive medical data through communication with a database within a hospital environment, a cloud server that performs tasks such as the standardization of medical data, a client such as a smart watch, a medical computing device, or the like. The network unit 130 may transmit the output data of a machine learning model, and the intermediate data, processed data, and like derived from the computational process of the processor 110 through communication with the above-described database, server, client, or computing device.

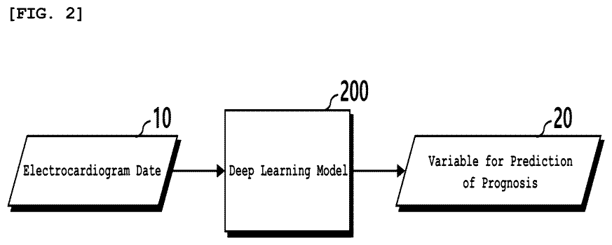

FIG. 2 is a block diagram showing the analysis process of a deep learning model according to one embodiment of the present disclosure.

Referring to FIG. 2, a deep learning model 200 according to one embodiment of the present disclosure may receive electrocardiogram data 10 and output a variable 20 for the prediction of the prognosis of a patient whose electrocardiogram data 10 was measured. In this case, the patient whose electrocardiogram data 10 was measured may be a heart failure patient. Furthermore, the variable 20 for the prediction of the prognosis may be the patient's mortality rate within n years from the time when the electrocardiogram data was measured. That is, the deep learning model 200 according to the one embodiment of the present disclosure may analyze the probability that the heart failure patient whose electrocardiogram data 10 was measured will die within n years and output the variable 20 for the prediction of the prognosis.

The deep learning model 200 may be composed of a combination of a stem block, residual blocks, and a fully connected network. For example, the deep learning model 200 may be composed of one stem block, four residual blocks, and one fully connected network. Each of the blocks may include layers such as a one-dimensional convolutional neural network (Conv1D), batch normalization (BatchNorm1d), rectified linear unit activation (ReLU), and a dropout layer. Furthermore, only the stem block and the first layer may have skip connections with max pooling (MaxPool1d).

In the following, there will be described the results of a study that validated a deep learning model for predicting one-year mortality in heart failure patients with reduced ejection fraction, which had the structure of the example described above.

### (1) Study Design and Population

A multicenter retrospective cohort study was conducted at two South Korean hospitals to develop and validate a deep learning model for predicting one-year all-cause mortality in patients having heart failure with reduced ejection fraction. The analysis was based on high-quality electrocardiogram data extracted from 3,894 patients collected between September 2016 and May 2021. Patients who had an ejection fraction (EF) of 40% or less and completed one-year follow-up were targets for the study. The study interval between electrocardiographic and echocardiographic tests was limited to 14 days before and after the two procedures. Electrocardiograms were labeled based on the mortality events that occurred during a follow-up period. The Institutional Review Boards (IRBs) of the two hospitals approved this study, and informed consent was waived due to the retrospective nature of the study, fully anonymized dataset, and minimal risk to patients.

### (2) Data Collection and Results

Electrocardiogram data was extracted at a sampling rate of 500 Hz from the two hospitals and stored in the MUSE Cardiology Information System. Supplementary patient demographic and clinical data, including EF data, was obtained from electronic medical records. The EF data was determined using a biplane approach together with the Simpson method. When one or more echocardiograms were obtained within 14 days after the electrocardiogram test, an echocardiogram closest to an electrocardiogram was used as an index echocardiogram. Clinical data, including age, sex, diabetes, hypertension, chronic kidney disease, and atrial fibrillation/atrial flutter, was obtained from the hospitals.

The primary result of this study was the prediction performance of the deep learning model for predicting one-year mortality. All-cause mortality data was cross-checked with national official death certification data.

### (3) Development of Deep Learning Prediction Model

The structure of the deep learning model for predicting the one-year mortality of patients having heart failure with reduced ejection fraction is the same as the structure of the deep learning model 200 described above, so that a detailed description thereof is omitted. The Adam optimizer, the focal loss function, and the cosine warm-up scheduler were used to train the deep learning model. Singles were normalized through preprocessing for electrocardiograms, and down-sampling was performed to reduce the sampling rate from 500 Hz to 250 Hz. Furthermore, transformation for data augmentation was applied.

### (4) Statistical Analysis

For the validation process, a deep learning model was used to convert each internal data input (ECG) into a binary representation representing one-year mortality in the range from 0 (non-survival HFrEF) to 1 (survival HFrEF). The area under the receiver operating characteristic curve (AUROC) was used to evaluate the performance of the model. However, in this analysis, the accomplishment of a high negative predictive value (NPV) was prioritized. The driving force was the clinical goal of identifying HFrEF patients with a probability of surviving for more than one year. Predicting survival is critical in the context of heart failure. A high NPV helps to better identify patients who may not require interventions such as implantable cardioverter defibrillators (ICDs).

By taking into consideration this, the highest negative predictive value was calculated by setting the optimal cutoff value for each procedural factor to determine the sensitivity, specificity, positive predictive value (PPV), and especially negative predictive value of the model. The optimal cutoff value for each procedural factor for the prediction of one-year mortality was determined using Youden's J statistic. The point where the sensitivity reached 0.99 in the training dataset was set as the optimal cutoff value to meet the consensus among researchers who prioritized sensitivity in clinical decision making.

Furthermore, this analysis included cumulative event analysis that was performed through estimation using Kaplan-Meier curves and comparison using log-rank tests. The Cox proportional hazards model was used to calculate hazard ratios (HRs) and 95 % confidence intervals (CIs) for independent predictors of one-year mortality. The covariates used in the analysis were selected based on whether there was a significant difference (p-value<0.1) between the two groups or there was a predictive value. Age, gender, body mass index, the previous diagnosis of diabetes, hypertension, and chronic kidney disease, whether optimal treatment has been received, and the high-risk/low-risk classification of the deep learning model were incorporated into the Cox proportional hazards regression model. Finally, sensitivity maps were generated to highlight key aspects that influenced the developed deep learning model. All analyses were performed using the R Foundation for statistical computing.

### (5) Results

This study included a total of 3,894 HFrEF patients (mean age: 64.3 years; mean electrocardiogram: 29.8%) and 16,228 electrocardiograms. The samples consisted of 63.6% males (2,478), 30.3% hypertension patients (1,179), 28.3% diabetes patients (1, 103), and 5.1% chronic kidney disease patients (199). The one-year mortality was 8.7% (339 persons out of 1, 660 electrocardiogram patients).

A brief observation showed that the group of patients who died within a year of diagnosis was generally older and had lower diastolic blood pressure, higher heart rate, lower ejection fraction, and higher prevalence of hypertension, diabetes, chronic kidney disease, and atrial fibrillation. Furthermore, the number of patients who received optimal treatment was lower in this group. In this study, the definition of the optimal medical treatment included patients who were concurrently using a beta-blocker, a renin-angiotensin system inhibitor (RASI), and a mineralocorticoid receptor antagonist (MRA). It is noteworthy that an angiotensin receptor-neprilysin inhibitor (ARNI) was covered by insurance from 2018 and a sodium-glucose cotransporter-2 (SGLT2) inhibitor was approved for use in heart failure in Korea in 2022. This might had led to a decrease in the number of patients who received these drugs in the dataset.

The performance of the deep learning model (DLM) was evaluated as 0.826(95% CI, 0.794-0.859) in the test set by using the area under the receiver operating characteristic curve (AUROC). The sensitivity, specificity, positive predictive value, and negative predictive value scores of this model were 99.0%, 11.7%, 16.6%, and 98.4%, respectively.

The Cox regression model was used identify independent predictors of one-year mortality. As a result of adjusting for covariates, belonging to a high-risk group according to the deep learning model, being 65 years or older, chronic kidney disease, hypertension, atrial fibrillation/atrial flutter, and being male significantly increased the risk of death within one year. In particular, as a result of adjusting for covariates such as age, gender, and various underlying diseases, belonging to the high-risk group according to the deep learning model was found to be the strongest predictor of mortality with a hazard ratio of 4.12 (95% CI, 2.32-7.33, p<0.001). Furthermore, being 65 years or older, chronic kidney disease, hypertension, atrial fibrillation/atrial flutter, and being male were associated with an increased risk of death with hazard ratios of 2.93, 1.89, 1.50, 1.21, and 1.20, respectively (all p<0.001). In contrast, when optimal medical treatment was received, the risk of death was found to decrease to 0.53 (95% CI, 0.48-0.59, p<0.001). According to the Kaplan-Meier estimate of mortality, the group classified as the high-risk group according to the deep learning model had a significantly higher mortality.

To better understand the capabilities of the deep learning model, there were visualized the electrocardiogram regions that the deep learning model focused on when identifying patients with a high one-year risk of death in HFrEF patients by using sensitivity maps. Interestingly, the deep learning model was found to focus more on ST segments of the V1 and V3 leads than on QRS complexes of other leads. In other words, it can be seen that the deep learning model performs prediction while providing weight to ST segments of at least one of the V1 and V3 leads in electrocardiogram data.

### (6) Discussion

Two primary observations were made: First, the proposed deep learning model showed strong predictive ability as being incorporated into the AUROC of 0.826. Second, as a result of adjusting for covariates, the model effectively identified high-risk patients with an HR of 4.12. When these results are taken together, the results support the potential of the analysis of the deep learning model for an innovative tool to meliorate the prognosis and risk stratification of HFrEF patients.

### (7) Clinical Implications of Deep Learning Model of Present Disclosure

The ability of the model of the present disclosure to stratify patients as a ‘high-risk’ group, whose one-year mortality is 4.12 times higher than that of a ‘low-risk’ group, outperforms existing prognostic indicators such as chronic kidney disease. This stratification may serve as an important prognostic factor in the management of HFrEF.

Furthermore, the high negative predictive value of the model of the present disclosure provides an efficient tool for predicting patients who are likely to die within one year, allowing clinicians to effectively prioritize resources for high-risk patients. Furthermore, the prognostic value of the model of the present disclosure may help identify patients who may benefit from an intervention such as an implantable cardioverter-defibrillator (ICD) or an intensive drug therapy.

The model of the present disclosure essentially improves the precision of risk stratification of heart failure disease, thereby providing new insights into relevant prognostic factors. This may open the way to improving heart failure management and patient treatment.

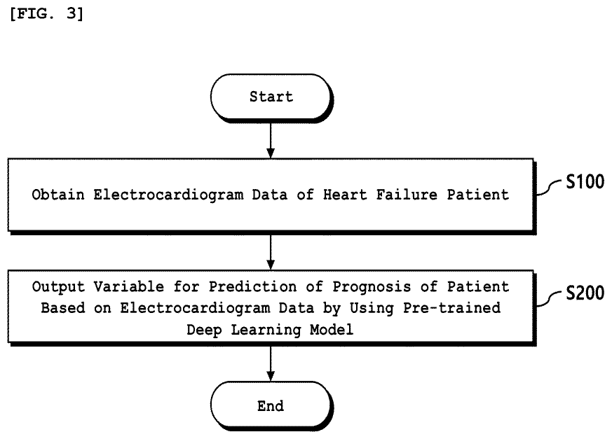

FIG. 3 is a flowchart showing a method of predicting the prognosis of heart failure according to one embodiment of the present disclosure.

Referring to FIG. 3, the computing device 100 according to one embodiment of the present disclosure may obtain electrocardiogram data of a heart failure patient in step S100. For example, when the computing device 100 is a client such as an electrocardiogram measurement device, the computing device 100 may generate electrocardiogram data by measuring an electrocardiogram signal of the heart failure patient. When the computing device 100 is a server, the computing device 100 may receive the electrocardiogram data through wired/wireless communication with the electrocardiogram measurement device.

The computing device 100 may output a variable for the prediction of the prognosis of the patient based on the obtained electrocardiogram data by using a pre-trained deep learning model in step S200. In this case, the variable for the prediction of the prognosis may be the mortality of the patient within n years from the time when the electrocardiogram data was measured. Furthermore, n years may be determined based on user input. When the user enters a desired period, the computing device 100 may predict the mortality of the patient within the entered period through the deep learning model. Meanwhile, the deep learning model may receive clinical data of the patient along with the electrocardiogram data, and may output a variable for the prediction of the prognosis of the patient. In this case, the clinical data may be a predictor selected through Cox regression analysis in the process of training the deep learning model. For example, the clinical data may include at least one of pieces of information about age, gender, body mass index, blood pressure, heart rate, ejection fraction, whether a chronic disease has been diagnosed, and whether optimal treatment has been performed. In this case, optimal treatment may be understood as a treatment that is clinically confirmed to be suitable for the treatment of heart failure.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1.-10. (canceled)

11. A computer-implemented method of facilitating prognosis of heart failure based on signal processing analysis of electrocardiogram signal data, the method comprising:
accessing, by a computer system, electrocardiogram (ECG) signal data obtained from one or more leads attached to a subject;
inputting, by the computer system, the accessed ECG signal data into a deep learning model, the deep learning model trained by:
accessing training data obtained from a plurality of other subjects, the training data comprising ECG signal data and heart failure data;
analyzing the ECG signal data obtained from one or more leads attached to the plurality of other subjects;
analyzing the heart failure data obtained from the plurality of other subjects; and
training the deep learning model based on correlations between the analyzed ECG signal data obtained from the plurality of other subjects and the analyzed heart failure data obtained from the plurality of other subjects; and
generating, by the computer system, a prediction of prognosis of heart failure of the subject derived from an output of the deep learning model, wherein the deep learning model is configured to generate an output based at least in part on analyzing the accessed ECG signal data obtained from the one or more leads attached to the subject,
wherein the computer system comprises a computer processor and an electronic storage medium.

12. The computer-implemented method of claim 11, wherein analyzing the accessed ECG signal data comprises assigning different weights to one or more ST segments or QRS complexes of the accessed ECG signal data.

13. The computer-implemented method of claim 12, wherein ST segments of V1 and V3 leads are more heavily weighted compared to QRS complexes of other leads.

14. The computer-implemented method of claim 11, wherein the heart failure data comprises timing of heart failure.

15. The computer-implemented method of claim 11, wherein the deep learning model is further trained by:
repeatedly calculating an error rate by cross-comparing the output against data derived from death certificates; and
repeatedly updating neural network parameters constituting the deep learning model until the calculated error is within a predetermined threshold.

16. The computer-implemented method of claim 11, wherein the deep learning model is trained based on one or more of supervised learning, semi-supervised learning, or unsupervised learning.

17. The computer-implemented method of claim 11, wherein the deep learning model comprises one or more stem blocks, one or more residual blocks, and a fully connected network.

18. The computer-implemented method of claim 17, wherein the deep learning model comprises one stem block, four residual blocks, and one fully connected network.

19. The computer-implemented method of claim 17, wherein each of the one or more stem blocks and the one or more residual blocks comprise one or more convolutional neural networks, batch normalization, rectified linear unit activation, or dropout layers.

20. The computer-implemented method of claim 11, further comprising generating, by the computer system, a graphical user interface comprising a visualization of the generated prediction of prognosis of heart failure of the subject.

21. The computer-implemented method of claim 11, wherein the heart failure comprises heart failure with reduced ejection fraction.

22. The computer-implemented method of claim 11, wherein the prediction of prognosis of heart failure of the subject comprises an expected mortality rate of the subject due to heart failure within a specific period of time.

23. The computer-implemented method of claim 11, further comprising:
accessing, by the computer system, clinical data of the subject; and
inputting, by the computer system, the clinical data of the subject into the deep learning model,
wherein the deep learning model is further configured to generate the output based at least in part on the clinical data.

24. The computer-implemented method of claim 23, wherein the clinical data comprises ejection fraction (EF) data determined using Simpson's biplane method.

25. The computer-implemented method of claim 23, wherein the clinical data comprises one or more of age, gender, body mass index, blood pressure, heart rate, ejection fraction, diagnosis of chronic disease, or treatment.

26. The computer-implemented method of claim 11, further comprising performing risk stratification to determine whether the subject is high-risk based at least in part on the generated prediction of prognosis of heart failure.

27. The computer-implemented method of claim 11, further comprising determining customized treatment for the subject based at least in part on the generated prediction of prognosis of heart failure.

28. A system for facilitating prognosis of heart failure based on signal processing analysis of electrocardiogram signal data, the system comprising:
one or more computer readable storage devices configured to store a plurality of computer executable instructions; and
one or more hardware computer processors in communication with the one or more computer readable storage devices and configured to execute the plurality of computer executable instructions in order to cause the system to:
access electrocardiogram (ECG) signal data obtained from one or more leads attached to a subject;
input the accessed ECG signal data into a deep learning model, the deep learning model trained by:
accessing training data obtained from a plurality of other subjects, the training data comprising ECG signal data and heart failure data;
analyzing the ECG signal data obtained from one or more leads attached to the plurality of other subjects;
analyzing the heart failure data obtained from the plurality of other subjects; and
training the deep learning model based on correlations between the analyzed ECG signal data obtained from the plurality of other subjects and the analyzed heart failure data obtained from the plurality of other subjects; and
generate a prediction of prognosis of heart failure of the subject derived from an output of the deep learning model, wherein the deep learning model is configured to generate an output based at least in part on analyzing the accessed ECG signal data obtained from the one or more leads of the subject.

29. The system of claim 28, wherein analyzing the accessed ECG signal data comprises weighting ST segments of V1 and V3 leads more heavily compared to QRS complexes of other leads.

30. The system of claim 28, wherein the deep learning model is further trained by:
repeatedly calculating an error rate by cross-comparing the output against data derived from death certificates; and
repeatedly updating neural network parameters constituting the deep learning model until the calculated error is within a predetermined threshold.
